(19) United States
(12) Patent Application Publication
(10) Pub. No.: US 2019/0125297 A1
(43) Pub. Date: May 2, 2019

(54) DEVICE FOR IMAGING ASSISTED MINIMALLY INVASIVE IMPLANT AND JAWBONE RECONSTRUCTION SURGERY

(71) Applicant: THE REGENTS OF THE UNIVERSITY OF MICHIGAN, Ann Arbor, MI (US)

(72) Inventors: Hsun-Liang Chan, Ann Arbor, MI (US); Oliver D. Kripfgans, Ann Arbor, MI (US); J. Brian Fowlkes, Ann Arbor, MI (US)

(21) Appl. No.: 16/092,030

(22) PCT Filed: Apr. 7, 2017
(86) PCT No.: PCT/US17/26528

§ 371 Date: Oct. 8, 2018

Related U.S. Application Data
(60) Provisional application No. 62/319,932, filed on Apr. 8, 2016.

(51) Int. Cl. A61B 8/08; A61B 8/00; A61C 9/00; A61B 34/10; A61C 13/00; A61B 8/12

(52) U.S. Cl. CPC A61B 8/0875; A61B 8/4455; A61B 8/4477; A61C 9/0086; A61B 8/483; A61B 2090/3925; A61B 8/4483; A61B 8/4281; A61B 34/10; A61C 13/0004; A61B 8/12; A61B 8/4461

(57) ABSTRACT

A device for 3-D imaging of the oral cavity for purposes of implant positioning, and in particular, a dental ultrasound scanner connected to a registration device that provides coordinates for realigning ultrasound images. The ultrasound scanner includes transducers having frequencies of 18 megahertz or higher and wavelengths of 80 microns or less. The reference device may be used as a surgical guide or a separate surgical guide may be created.

### CROSS-REFERENCE TO RELATED APPLICATIONS

This application is the U.S. national phase of International Application No. PCT/US17/26528, filed Apr. 7, 2017, which claims the benefit of the filing date of U.S. Provisional Application No. 62/319,932, filed Apr. 8, 2016, which is hereby incorporated by reference in its entirety.

### FIELD OF THE DISCLOSURE

This disclosure relates generally to a device for 3-D imaging of the oral cavity for purposes of evaluating oral anatomical structures, implant positioning, and implant related procedures such as jawbone reconstruction surgery. In particular, this disclosure relates to a dental ultrasound scanner, a registration device that provides coordinates, and computer algorithm for realigning ultrasound images and facilitates the placement of implants.

### BACKGROUND

Despite advances in dental care, millions of people suffer tooth loss caused by tooth decay, gum disease, or injury. For many years, the only treatment options available for people with missing teeth were bridges and dentures. Today, dental implants are available. Dental implants are essentially replacements for missing teeth. They provide a strong foundation for fixed or removable replacement teeth that are made to match the natural teeth. The number of implants conducted in the United States is rapidly growing, and estimates suggest that approximately two to four million dental implants will be placed in the United States by 2020.

Implant dentistry generally involves the use of a hand-piece that is guided freehand when drilling the implant osteotomy. The surgeon transfers the implant positions, normally preplanned on a radiograph and/or dental model, into the jaw of the patient. However, the transference of implant planning is not always met because of limitations to human perception and the ability to correlate image data to surgical sites without suitable aids. Navigation systems are increasingly being used in oral implantology that assist surgeons in measuring and visualizing accurately the precise and current position of an instrument relative to the anatomy of the patient. This means that an implant position can be transferred to the jaw according to the image-supported design with a certain degree of precision. Image-guided surgery allows for axis-parallelism of the implants and can be performed with a minimal amount of invasive surgery while avoiding damage to sensitive structures.

When planning and performing surgery in the oral cavity, a dentist or surgeon has to know the spatial relationships of jaw structures, e.g. soft tissue, bone, vital structures, in 3 dimensions. Currently three-dimensional images of the jaw are obtained from cone-beam computed tomography (CBCT). However, CBCT emits radiation, is expensive, and is not convenient. It might not be used during the surgery to provide surgical feedback because of accumulated radiation and lack of access to the CBCT scanner. Additionally, the quality of CBCT scan is greatly reduced by the presence of highly radiation-reflective structures, e.g. metal restorations and implants.

Ultrasound can be a very useful imaging tool to show images of the jawbone surface structures and some specific vital structures, such as the mental nerve, the greater palatine nerve, and the lingual nerve. It is non-ionizing, less expensive, real-time, and can be used easily within the surgery room. However, all current available ultrasound transducers can only provide images of part of the jawbone surface in 2 dimensions, e.g. buccal, lingual or crestal side of the jawbone surface. These images cannot tell the bucco-lingual dimension of the jawbone. In other words, a device and method that can easily adapt to the current existing implant surgery work flow to obtain a 3 dimensional ultrasound data set, including all the buccal, lingual and crestal sides is currently lacking.

### SUMMARY OF THE DISCLOSURE

The current disclosure is directed to an ultrasound imaging device that can aid in producing three-dimensional images of the oral cavity from which implant procedures can be planned with software and a surgical guide made with, but not limited to rapid prototyping technology. The device includes a dental ultrasound scanner that can scan the jawbone of a patient to provide a series of buccal and lingual images of the jawbone and a registration device that may have a dual function of providing coordinates for realigning ultrasound images and facilitating the placement of implants. The two sets of images (buccal and lingual) provided by the dental ultrasound scanner are merged by the registration device into dual-sided compound images, which are subsequently three-dimensionally reconstructed to provide feedback regarding implant positioning and other implant related procedures.

The ultrasound scanner is tailored to ergonomically fit oral anatomies. The ultrasound scanner includes at least one high resolution transducer provided on a probe. The transducer operates at or above 18 megahertz and have a wavelength at or below 80 micron. The probe is small enough to be comfortable in the mouth, having a length around 20 mm and an aperture for the transducer of approximately 13 mm. The probe may come in different sizes to accommodate differences in the size of oral cavity. The probe may also be used outside or inside the mouth of a patient. In some embodiments within the scope of the present disclosure, two transducers and 2D arrays as well as mechanically swept arrays may be provided so that buccal and lingual images can be provided nearly simultaneously. The use of more than two arrays may be facilitated to work together to form a 1D to 4D image, that is up to 3D spatial dimensions plus a temporal dimension.

The registration device may be secured within the mouth of a patient at the location of an implant. For example, the registration device may be cube-shaped and secured by extensions to the occlusal surfaces of neighboring teeth, like a bite guard, for the stability of the registration device. The patient can wear the registration device during ultrasound scans so that the spatial relations between the ultrasound probe, the registration device and oral anatomies can be recorded. The registration device includes internal landmarks that allow for 3D imaging via triangulation. The registration device may be modified ergonomically to fit various oral anatomies. On reconstructed 3D images, implant positions can be planned and the information about implant positions can be built into the cube with software.

During the implant surgery, the registration device may be used as a guide for implant placement. Alternately, a separate guide may be created by three-dimensional printing or other manufacturing methods. The guide, whether the registration device or a separate guide, may be made from acoustically penetrable material.

The imaging device can be used to provide surgical feedback by recording the implant drill position in relation to bone surfaces so the implant will not be placed unintentionally outside of the bone, which is a common complication for minimally invasive implant surgery. The imaging device may be used in situations where precise implantation plans are required, such as in situations with anatomical limitations, little space, or atrophic jawbone.

The imaging device of the present disclosure may also have uses unrelated to implant surgery. For example, the imaging device may be used to detect caries with greater reliability than CT scans, measure intraoral soft tissue dimensions, identify oral cancer, diagnose metastasis to cervical lymph nodes, and diagnose postoperative stitch abscesses, cystic lesions, benign tumors, malignant tumors, lyphadenopathies, and other abscesses. In addition, the imaging device may be used to reconstruct three-dimensional surface images of periodontal structures and defects.

### BRIEF DESCRIPTION OF THE DRAWINGS

FIG. 1 illustrates a mouth with a missing tooth on which the imagining device of the present disclosure may be used;

FIG. 2 illustrates a registration device of the imagining device of the present disclosure;

FIG. 3 illustrates a probe of an ultrasonic scanner of the imagining device of the present disclosure;

FIG. 4 illustrates a horseshoe connector of the imaging device of the present disclosure;

FIG. 5 illustrates a track system for the imaging device of the present disclosure;

FIG. 6 illustrates a track system on a mouth tray for the imaging device of the present disclosure;

FIG. 7 illustrates a 3-D ultrasound image reconstructed from buccal and lingual scans by the ultrasound scanner;

FIG. 8 illustrates a virtual plan for implant created on the 3-D ultrasound image;

FIG. 9 illustrates the registration device of the imaging device converted into a guide for implant surgery;

FIG. 10 illustrates a reference pin or object of the imaging device of the present disclosure;

FIG. 11 illustrates a surgical drill inserted at the planned location of a patient's mouth;

FIG. 12 illustrates an implant placed in the planned location after the surgical drill is used; and

FIG. 13 illustrates landmarks within a registration device used for orientation of ultrasound images in space.

### DETAILED DESCRIPTION

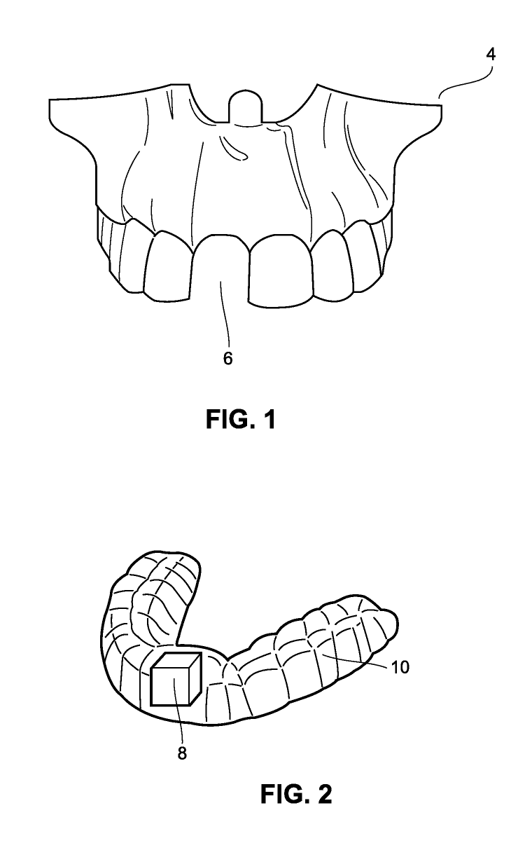

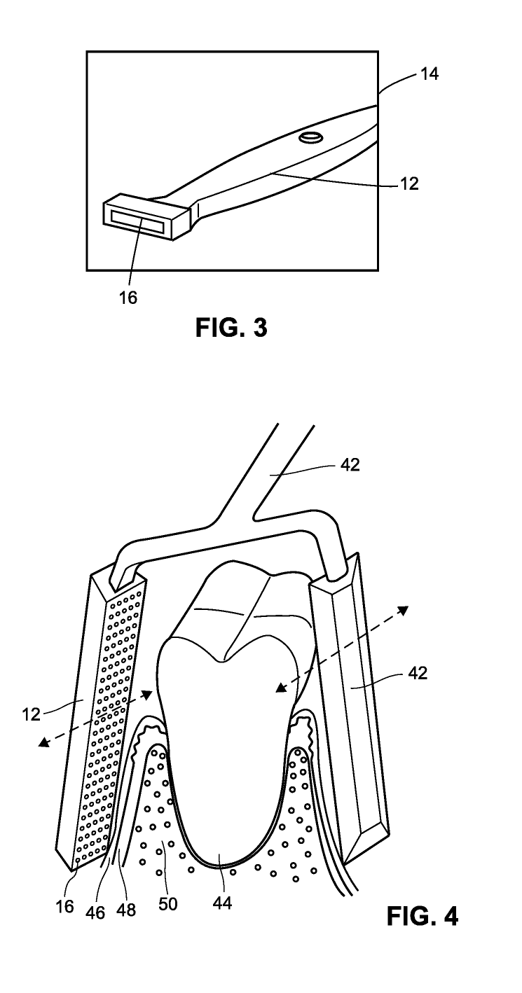

FIGS. 1-10 depict a proposed workflow of an embodiment of an imaging device 2 of the present disclosure. FIG. 1 depicts the mouth 4 of a patient having a missing tooth 6 (by way of example only, a missing front tooth) and requiring an implant. FIG. 2 depicts a reference device 8 made with the aid of a study model of the patient's maxillary teeth. The reference device 8 is cube-shaped with extensions 10 to secure the registration device 8 in place. The reference device 8 is designed so that the cube-shaped portion sits where the tooth 6 is missing.

FIG. 3 depicts a probe 12 of an ultrasound scanner 14. The probe 12 is ideally sized to fit comfortably into the mouth of a patient. In some embodiments within the scope of the present disclosure, the probe 12 has a width of 20 mm, with a 13 mm aperture for at least one transducer 16. In some embodiments within the scope of the present disclosure, two probes 12 may be used to conduct ultrasound scans, one on the buccal side of the patient's jawbone and one on the lingual side of the patient's jawbone. In some embodiments within the scope of the present disclosure, the probes 12 may be mounted to the fingers of a medical practitioner rather than provided on a toothbrush-like device.

In some embodiments within the scope of the present disclosure, the ultrasound scanner 14 includes a pair of freestanding and registered 1-D or 2-D array transducers 16. Use of 2-D transducers 16 lowers the number of landmarks required within the registration device and may provide a faster and higher spatial resolution. Transducers 16 within the scope of the present disclosure include CMUTs (capacity micromachined ultrasound transducers), including CMOS realization, and PMUTs (piezoelectric micromachined ultrasound transducers), as well as other current or traditional designs. Such transducer designs, including CMOS, benefit from local electronics and naturally allow for miniaturization. The transducers 16 must have a high resolution, preferably having frequencies of 18 megahertz or higher and wavelengths of 80 microns or less. Lower frequencies may be used as well where lower spatial resolutions are allowable.

The transducers 16 may be located on separate probes 12 or they may be connected by a rigid horseshoe connector 42 as depicted in FIG. 4. The horseshoe connector 42 connects probes 12 located on the opposite side of a tooth 44, near the gum epithelium 46 and over the gum connective tissue 48 and jawbone 50. Movement of the probes 12 may be motorized in some embodiments within the scope of the present disclosure. A horseshoe connector 42 provides the advantage that the relative spatial location of the transducers 16 is known, which reduces the number of landmarks required within a registration device. Additionally, higher spatial resolution may be possible using a horseshoe connector 42. Concurrent imaging is possible using a horseshoe connector 42, which reduces scan time and therefore allows for faster procedures.

The probes 12 may include a coupling medium that eliminates the need to apply a gel-based medium on the area to be scanned. The coupling medium for the probes 12 may be water bags or jets, gel pads, or other known solutions in the art. The coupling medium is used to allow for maximum acoustic energy transfer from the probe 12 into the gum tissue and back, by removing or minimizing air interfaces. Air pockets and layers attenuate and reflect acoustic energy, thereby reducing the achievable depth penetration, signal to noise and contrast to noise ratios for best possible image clarity.

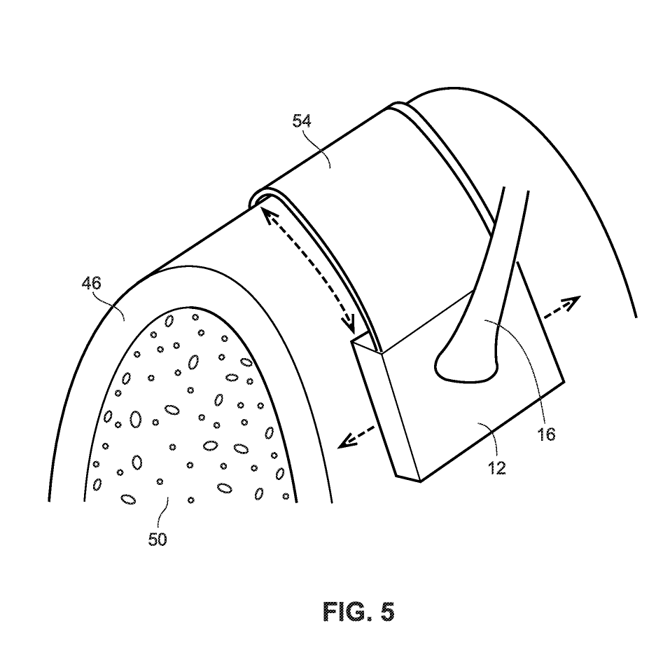

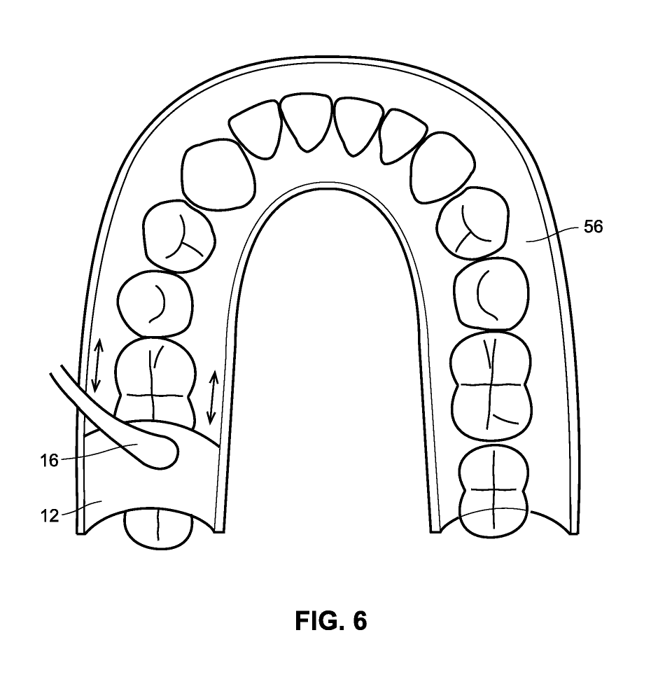

FIG. 5 illustrates a track or guide 54 upon which a probe 12 may travel. The track 54 may be configured to allow a 1-D or 2-D array probe across part of the jawbone 50 faciolingually and/or mesiodistally. The track 54 may be made of an acrylic and/or composite material that is adapted to the shape of the jawbone. As shown in FIG. 6, a probe 12 may alternately travel on a tray 56, which could be made after a tooth mold in the shape of the jawbone from an acrylic or composite material. The probe 12 may then be capable of sweeping along the whole jawbone until completion of the scan.

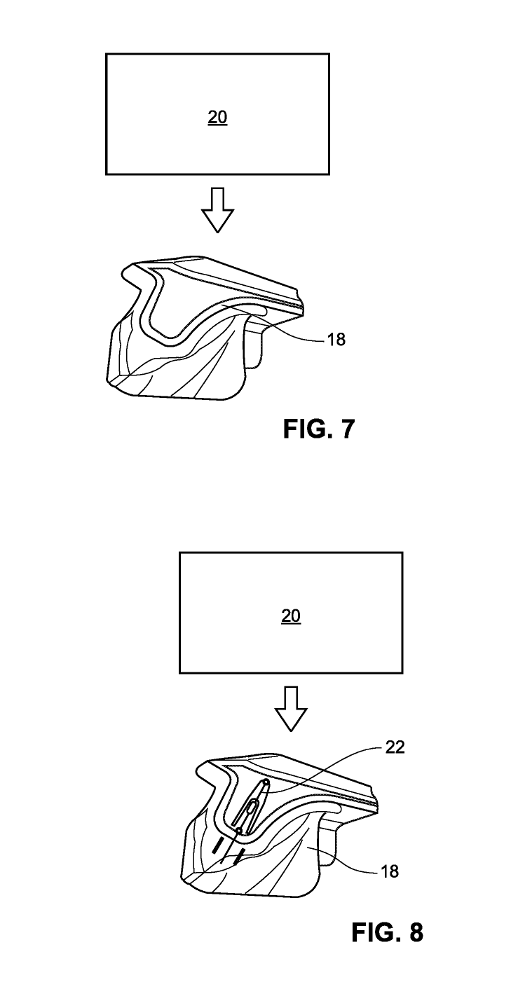

FIG. 7 depicts a 3-D ultrasound image 18 reconstructed from the buccal and lingual scans conducted by the ultrasound scanner 14. The figure shows a cross-sectional view. A computer processor 20 and algorithm may be used to create the 3-D ultrasound image 18. Additional information about the image-processing algorithm is discussed with respect to FIG. 12.

FIG. 8 depicts a plan 22 for implant placement constructed in conjunction with the 3-D ultrasound image 18. Software stored on a non-transitory computer readable medium associated with the computer processor 20 is used to model how the implant will be placed into the edentulous ridge or other desired location. The software may overlay the 3-D ultrasound image 18 with other images, such as CBCT images, for purposes of planning and performing oral surgeries such as periodontal surgeries, implant placement surgeries, bone augmentation procedures, and soft tissue grafting. In some embodiments within the scope of the present disclosure, the software can automatically or with manual assistance identify and color jaw structures, such as a tooth surface, root surface, jawbone surface, or soft tissue surface. The software may also be able to measure the dimension of jaw structures and distances between the jaw and other related structures in three dimensions.

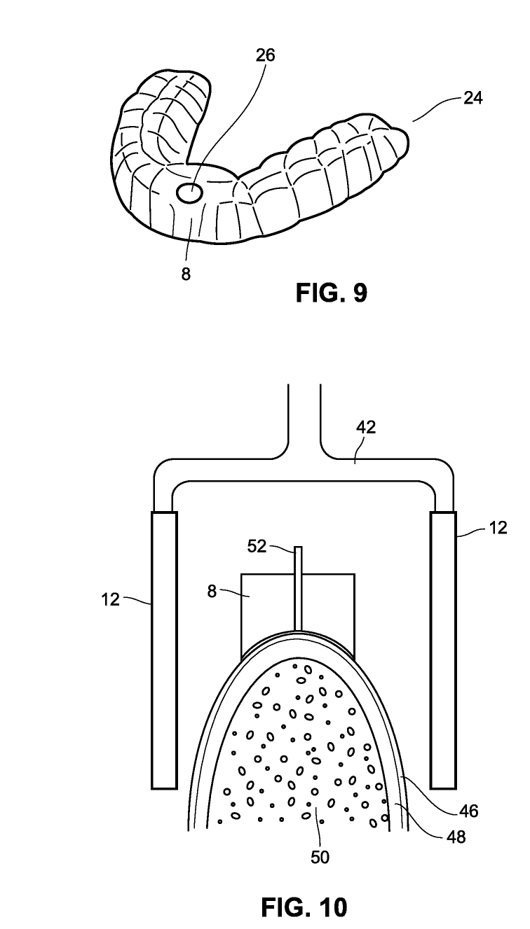

FIG. 9 depicts a surgical guide 24 created from the reference device 8 after information about implant positioning is built into the reference device 8. The guide hole 26 shown on the guide 24 is for insertion of surgical drills. In other embodiments within the scope of the present disclosure, a surgical guide 24 may be created that is separate from the reference device 8. In some embodiments within the scope of the present disclosure, the surgical guide 24 may be made of a rigid plastic having a low viscosity. If the viscosity of the surgical guide is too high, the viscous properties of the surgical guide 24 may interfere with the ultrasonic imaging. In some embodiments within the scope of the present disclosure, a non-viscous material may be used if coated by or embedded within a material that allows the necessary flow of ultrasonic waves via impedence matching.

FIG. 10 depicts a reference pin or object 52 affixed to the reference device 8 prior to surgery so that the planned implant position can be shown in relation to a jawbone 50 before the implant surgery. The relative position between the reference object 52 and the surface of the jawbone 50 is computed and displayed. With such information, planning of the drill-guide-hole for implant anchoring is performed. This ultrasound approach to planning is not only repeatable but can also be performed in situ when the surgical guide 24 is in place.

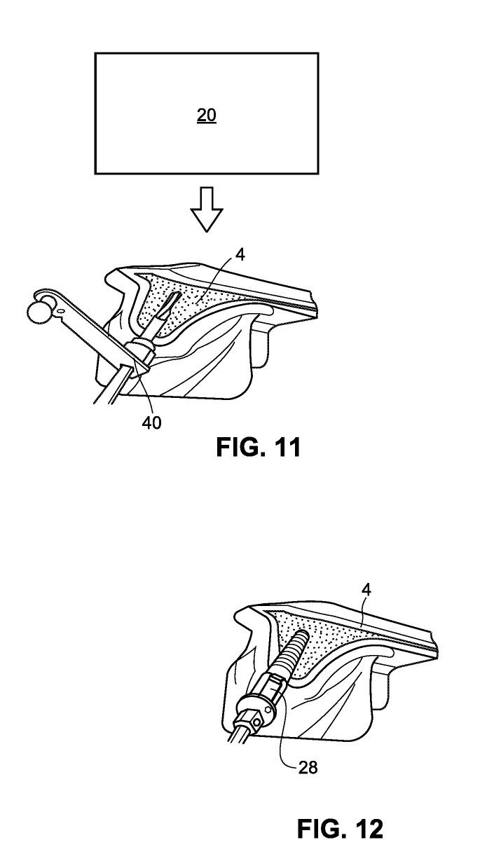

FIG. 11 depicts a surgical drill 40 being inserted at the planned location within the patient's mouth 4. During surgery, a computer processor 20 may overlay the pre-surgical 3-D ultrasound images with real-time images derived during the surgery to provide additional guidance for the surgery. The computer processor 20 may provide feedback to the surgeon about the surgery, for example notifying a surgeon placing bone grafts when the desired grafting volume is achieved. FIG. 12 depicts an implant 28 placed in the planned location of the patient's mouth 4 after drilling has been completed.

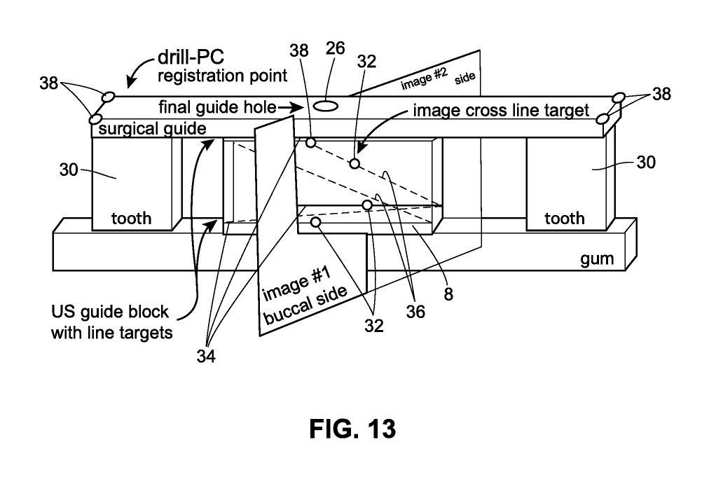

FIG. 13 depicts the registration device 8 located at a desired implant location between two teeth 30. Landmarks 32 are located on and within the registration device. The position of the landmarks 32 in ultrasound images from, for example, the buccal and lingual sides, can be mapped as a function of space using triangulation. Image processing algorithms can, for each ultrasound image I, derive the position of I in 3D space with respect to the landmarks 32. The landmarks create line targets on and within the registration device 8. In the embodiment depicted in FIG. 13, the solid lines 34 represent axes of translation and rotation. The dashed line 36 represents another axis of translation. Correlations between the straight and angled lines and landmarks 32 provide a functional relationship by which the image processing algorithm can derive the position of an image I in 3D space. Images that have had their position identified between, for example, between and \vec{rₙ} and \vec{rₘ}, respective normals \vec{nₙ} and \vec{nₘ} i.e. Iₙₘ can be merged to form a 3-D image space Sₙₘ. Two image spaces Sₙₘ¹ and Sₙₘ² can be taken on the buccal and lingual side of a dental anatomy of the interest as depicted in FIG. 13. The two image spaces Sₙₘ¹ and Sₙₘ² can then be co-related. Contained structures such as the jaw bone, for example, imaged from the lingual and buccal side, can be displayed in 3-D.

The registration device 8 depicted in FIG. 13 can be machined by use of the ultrasound image information alone or in conjunction with x-ray or CBCT imaging information. Machining will produce the guide hole 26 for drill placement and direction. Three-dimensional visualization of the jaw bone allows a surgical guide to be developed that ensures proper implant position and drilling direction. Machining-specific landmarks 38 on the surgical guide in FIG. 13 can be added after the initial ultrasound scanning to allow a machining device, such as a CNC machine, to register the registration device during the guide hole creation process. The machining device can register the machining-specific landmarks 38 or the original ultrasound landmarks 32.

1. An ultrasound imaging device comprising:
an ultrasound scanner including at least one probe containing a transducer, the transducer having a frequency of 18 megahertz or higher and wavelength of 80 microns or less;
a reference device comprising landmarks;
a processor containing image processing algorithms for deriving the position of 2-D images taken by the ultrasound scanner relative to the reference device and merging the 2-D images to form a 3-D image.

2. The ultrasound imaging device of claim 1, wherein the at least one probe has at least one of: a length of approximately 20 mm or an aperture of approximately 13 mm.

3. The ultrasound imaging device of claim 1, wherein the ultrasound scanner contains at least one of: a structure for being mounted on a finger, a track upon which the probe moves, and a tray upon which the probe moves.

4. The ultrasound imaging device of claim 1 comprising two probes, each probe comprising a transducer.

5. The ultrasound imaging device of claim 1, wherein at least two transducers are at least one of: connected by a connector at a known distance from one another, 2-D array transducers, or each of the at least two transducers are one of a capacity micromachined transducer and a piezoelectric micromachined ultrasound transducer.

6. The ultrasound imaging device of claim 1, wherein the at least one probe includes a coupling medium.

7. The ultrasound imaging device of claim 1, wherein the processor contains image overlay algorithms for overlapping the 3-D image with non-ultrasound images.

8. The ultrasound imaging device of claim 1, wherein the processor is associated with structure identification software stored on a non-transitory computer readable medium.

9. The ultrasound imaging device of claim 1, wherein the processor contains algorithms for measuring distances between structures identified by the structure identification software.

10. The ultrasound imaging device of claim 1, wherein the reference device includes at least one of: an extension for securing the device in the mouth, a guide hole, and machining-specific landmarks for creation of the guide hole.

11. The ultrasound imaging device of claim 1, wherein the reference device includes one of a rigid plastic having a low viscosity or an impedance-matching material composition.

12. The ultrasound imaging device of claim 1, further comprising a separate guide.

13. The ultrasound imaging device of claim 12, wherein the separate guide is a 3-D printed separate guide.

14. The ultrasound imaging device of claim 12, wherein the separate guide includes one of a rigid plastic having a low viscosity or an impedance-matching material composition.

15. The ultrasound imaging device of claim 1, further comprising a reference pin or object.
